(19) United States
(12) Patent Application Publication
(10) Pub. No.: US 2007/0208296 A1
(43) Pub. Date: Sep. 6, 2007

(54) Syringe Safety Device

(75) Inventors: John Paproski, North Wales, PA (US); Paul Norton, Trumbauersville, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: WEST PHARMACEUTICAL SERVICES, INC., Lionville, PA (US)

(21) Appl. No.: 11/747,773

(22) Filed: May 11, 2007

Related U.S. Application Data
(62) Division of application No. 11/059,729, filed on Feb. 17, 2005.

(51) Int. Cl. A61M 37/00; A61M 5/00

(52) U.S. Cl. 604/82; 604/187

(57) ABSTRACT

A tip cap for a syringe that limits contact between the syringe and a needle tip, wherein the needle tip penetrates the tip cap to one of dispense medication out of the syringe and draw medication into of the syringe. The syringe includes a luer cone extending from a barrel of the syringe. The tip cap includes a penetration wall and a generally cylindrical sleeve extending generally perpendicularly from the penetration wall adjacent a periphery of the penetration wall. The cylindrical sleeve includes a radially, inwardly extending locating shoulder on an inner surface that is positioned a predetermined distance from the penetration wall. The locating shoulder engages an end of the luer cone in an operating position. The penetration wall, cylindrical sleeve and luer cone define a receiving space within which the needle tip is positionable.

### CROSS-REFERENCE TO RELATED APPLICATIONS

This application is a division of U.S. patent application Ser. No. 11/059,729, filed Feb. 17, 2005.

### BACKGROUND OF THE INVENTION

The present application is directed to a syringe safety device for forming a fluid coupling between a sealed vial and a syringe. The syringe safety device preferably limits the exposure of a sharp needle to a user and, therefore, inadvertent pricks from the needle. The syringe safety device also provides a relatively simple process for mixing a diluent and a powdered medication to produce an injectable medication solution. It is often desirable to store drugs in a powdered form until immediately prior to administering the drug to a patient. Powdered medications typically have a longer shelf life than medications provided in a mixed or liquid solution form. Accordingly, powdered medications may be stored for a longer period of time and mixed with the diluent prior to use such that the shelf life of the medication is prolonged.

Several apparatus for transferring fluids between a vial and a syringe that generally protect a user from needle pricks have been developed. U.S. Pat. No. 6,729,370 ('370 patent) discloses a syringe safety device of this type and is incorporated herein by reference in its entirety. The syringe safety device of the '370 patent includes a connector 18 that is mountable to a vial 12 and a syringe 24 at opposing ends. A sliding joint 22 is slidably mounted in the connector 22. A needle 20 is mounted to the sliding joint for controlled sliding movement along a connector axis. The needle is typically encompassed by the connector during use or transfer of fluid to and from the syringe and vial to prevent inadvertent needle pricks. In operation, the syringe is mounted to the sliding joint and the vial is mounted to an opposing end of the connector. The syringe is urged toward the vial along the connector axis such that the needle penetrates a stopper 16 on the vial and a diluent is dispensed into the vial. The diluent and a powdered medication in the vial are mixed, resulting in a medication solution that is drawn through the needle and into the syringe. The syringe is removed from the sliding joint and a syringe needle is mounted on the distal end of the syringe so that the medication solution is injectable into the patient.

In operation, when the mixed solution has been drawn into the syringe, the syringe is removed from the connector and the sliding joint prior to engagement with the syringe needle. During this transition, the medication solution is exposed to an external environment at a syringe orifice where the medication solution could be contaminated. In addition, the medication may leak from the syringe orifice. Accordingly, it is desirable to cover the syringe orifice so that the medication solution is generally not exposed to an external environment through the orifice.

Further, when transferring a relatively large volume of fluid to or from the vial, a significant amount of pressure or a vacuum may be created up in the vial. Accordingly, air within the vial may push a vial stopper out of the vial and escape through the stopper or between a surface of the needle and the stopper. When the large volume of mixed medication is drawn out of the vial into the syringe, a vacuum may be created in the vial. This vacuum tends to draw atmospheric air into the vial through breaches in the stopper or at the penetration of the needle. The release and introduction of air into the vial while drawing and dispensing fluid to and from the syringe may expose the fluid to contaminants. It would be desirable for all air that enters or exits the vial to be filtered and, thereby, reduce the likelihood of contamination.

### BRIEF SUMMARY OF THE INVENTION

A preferred embodiment of the present invention is directed to a syringe safety device configured to form a fluid coupling between a sealed vial and a syringe having a barrel for receiving fluid. The syringe includes a luer cone extending from the barrel for dispensing or receiving fluid. The luer cone includes a distal end with a syringe orifice. The syringe safety device also includes a connector having a vial end and a syringe end. The syringe is movably mounted to the syringe end and the vial is mountable to the vial end. A tip cap is removably mounted to the luer cone of the syringe. The tip cap and the distal end define a receiving space therebetween. A needle is removably mounted to the connector and includes a needle tip facing toward the syringe in an assembled configuration. The needle tip is positioned in the receiving space when the connector and syringe are positioned in an engaged position.

In another aspect, the present invention is directed to a tip cap for a syringe that limits contact between the syringe and a needle tip, wherein the needle tip penetrates the tip cap to one of dispense medication out of the syringe and draw medication into of the syringe. The syringe includes a luer cone extending from a barrel of the syringe. The tip cap includes a penetration wall and a generally cylindrical sleeve extending generally perpendicularly from the penetration wall adjacent a periphery of the penetration wall. The cylindrical sleeve includes a radially, inwardly extending locating shoulder on an inner surface that is positioned a predetermined distance from the penetration wall. The locating shoulder engages an end of the luer cone in an operating position. The penetration wall, cylindrical sleeve and luer cone define a receiving space within which the needle tip is positionable.

In another aspect, the present invention is directed to a syringe safety device for transferring or mixing medications between a syringe and a vial. The syringe safety device includes a connector having a vial end and a syringe end. The syringe is removably mountable to the syringe end and the vial is mountable to the vial end. A tip cap is removably mountable to a luer cone of the syringe. At least one needle penetrates the tip cap in an engaged position and forms at least a portion of a fluid passageway between the syringe and vial. The tip cap is retained on the luer cone when a fluid has been transferred through the fluid passageway and the syringe is removed from the syringe end.

In yet another aspect, the present invention is directed to a method for transferring medication between a syringe having a tip cap mounted thereon and a vial. The method includes the steps of penetrating the tip cap with a needle to create a fluid passageway between the syringe and vial, drawing a medication from the vial, through the passageway and into the syringe and removing the needle from the tip cap. The tip cap is retained on the syringe when the needle is removed from the tip cap.

In another aspect, the present invention is directed to a method of distributing a syringe safety device having a syringe with a luer cone and a plunger, a vial and a fluid transfer assembly. The fluid transfer assembly includes a connector, a sliding piston and a sliding linkage. A needle and a spike are mounted to opposing ends of the sliding linkage. The method includes the steps of at least partially filling the syringe with a diluent, mounting a tip cap to the luer cone, shipping the syringe with the tip cap thereon to a remote location, assembling the syringe with the fluid transfer assembly and delivering the assembled syringe and fluid transfer assembly to an end user.

In another aspect, the present invention is directed to a method of mixing a diluent and a powdered medication using a fluid transfer assembly. The fluid transfer assembly includes a connector, a sliding piston and a sliding linkage with a needle and a spike. The fluid transfer assembly is assembled along a connector axis. The diluent is contained in a syringe having a tip cap mounted on a luer cone of the syringe and the powered medication is contained in a vial. The method includes the steps of mounting the syringe to a syringe end of the fluid transfer assembly such that the syringe is movable relative to the fluid transfer assembly, mounting the vial to a vial end of the fluid transfer assembly, moving the syringe a first distance along the connector axis in an engaging direction at least until the needle penetrates the tip cap and is in fluid communication with the diluent, moving the syringe a second distance along the connector axis in the engaging direction at least until the spike is in fluid communication with the vial creating a fluid passageway between the syringe and vial, dispensing the diluent into the vial through the sterile fluid passageway, mixing the diluent and powdered medication in the vial which results in a medication solution, drawing the medication solution through the sterile fluid passageway into the syringe and removing the syringe from the syringe end with the tip cap being mounted on the syringe.

In another aspect, the present invention is directed to a syringe safety device configured to form a fluid coupling between a sealed vial and a syringe. The syringe safety device includes a connector for receiving the sealed vial and syringe at opposite ends thereof. The connector defines a connector axis and a sliding linkage is movably mountable on the connector axis within the connector. A needle is mounted to a first side of the sliding linkage on the connector axis and a spike is mounted to a second side of the sliding linkage on the connector axis. The spike or the needle includes a first luer lock connector that releasably mounts to a second luer lock connector of the sliding linkage.

In another aspect, the present invention is directed to a syringe safety device configured to form a fluid coupling between a vial and a syringe. The syringe safety device includes a connector having a syringe end and a vial end. The connector defines a connector axis and the syringe is removably mountable to the syringe end. The vial is mountable to the vial end and a spike is movably mounted on the connector axis to the connector. A penetration end of the spike faces the vial end for piercing a stopper of the vial in an engaged position. The penetration end has a generally figure eight-shaped cross-sectional configuration.

In a further aspect, the present invention is directed to a syringe safety device configured to form a fluid coupling between a vial and a syringe. The vial includes a head defining a peripheral rim. The syringe safety device includes a connector having a syringe end and a vial end, wherein the connector defines a connector axis. The syringe is releasably mountable to the syringe end and the vial is mountable to the vial end. The connector has a generally cylindrical-shape and includes a lock window adjacent the vial end. A vial lock extends toward the connector axis into the lock window from a root end adjacent the vial end to a terminal end. The terminal end is in engagement with the peripheral rim when the vial is mounted to the vial end.

### BRIEF DESCRIPTION OF THE SEVERAL VIEWS OF THE DRAWINGS

The foregoing summary, as well as the following detailed description of the preferred embodiments of the present invention, will be better understood when read in conjunction with the appended drawings. For the purposes of illustrating the syringe safety device, there is shown in the drawings preferred embodiments of the present invention. It should be understood, however, that the invention is not limited to the precise arrangements and instrumentalities shown. In the drawings:

FIG. 1 is a perspective view of a syringe safety device in an assembled configuration in accordance with a first preferred embodiment of the present invention;

FIG. 2 is an exploded perspective view of the syringe safety device shown in FIG. 1;

FIG. 2A is a cross-sectional view of a tip of a spike of the syringe safety device shown in FIG. 2, taken along line 2A-2A of FIG. 2;

FIG. 2B is a cross-sectional view of the spike of the syringe safety device shown in FIG. 2, taken along line 2B-2B of FIG. 2;

FIG. 3 is a cross-sectional view of the syringe safety device shown in FIG. 1 in an initial position, taken along line X-X of FIG. 1;

FIG. 3A is a greatly magnified fragmentary, cross-sectional view of the syringe safety device shown in FIG. 1, taken from within the boundary of line 3A of FIG. 3;

FIG. 3B is a greatly magnified fragmentary, cross-sectional view of the syringe safety device shown in FIG. 1, taken from within the boundary of line 3B of FIG. 3;

FIG. 3C is a greatly magnified fragmentary, cross-sectional view of the syringe safety device shown in FIG. 1, taken from within the boundary of line 3C of FIG. 3;

FIG. 4 is a cross-sectional view of the syringe safety device shown in FIG. 1 in an intermediate position, taken along line X-X of FIG. 1;

FIG. 5 is a cross-sectional view of the syringe safety device shown in FIG. 1 in an engaged position, taken along line X-X of FIG. 1;

FIG. 6 is a partially exploded view of the syringe safety device shown in FIG. 1 with a luer-type needle mounted to an end of the syringe;

FIG. 7 is a greatly magnified cross-sectional view of a tip cap and a luer cone of the syringe safety device shown in FIG. 1;

FIG. 8 is a greatly magnified perspective view of a vial end of a connector of the syringe safety device shown in FIG. 1;

FIG. 9 is a greatly magnified perspective view of a double needle assembly in accordance with a second preferred embodiment of the syringe safety device of the present invention;

FIG. 10 is a cross-sectional view of the spike shown in FIG. 9, taken along line 10-10 of FIG. 9;

FIG. 11 is a greatly magnified perspective view of a vial end of a connector of the second preferred embodiment of the syringe safety device of the present invention; and

FIG. 12 is a perspective, cross-sectional view of the vial end of the connector shown in FIG. 11, taken along line 12-12 of FIG. 11.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” and “outwardly” refer to directions toward and away from, respectively, the geometric center of the syringe safety device and designated parts thereof. The terminology includes the above-listed words, derivatives thereof and words of similar import.

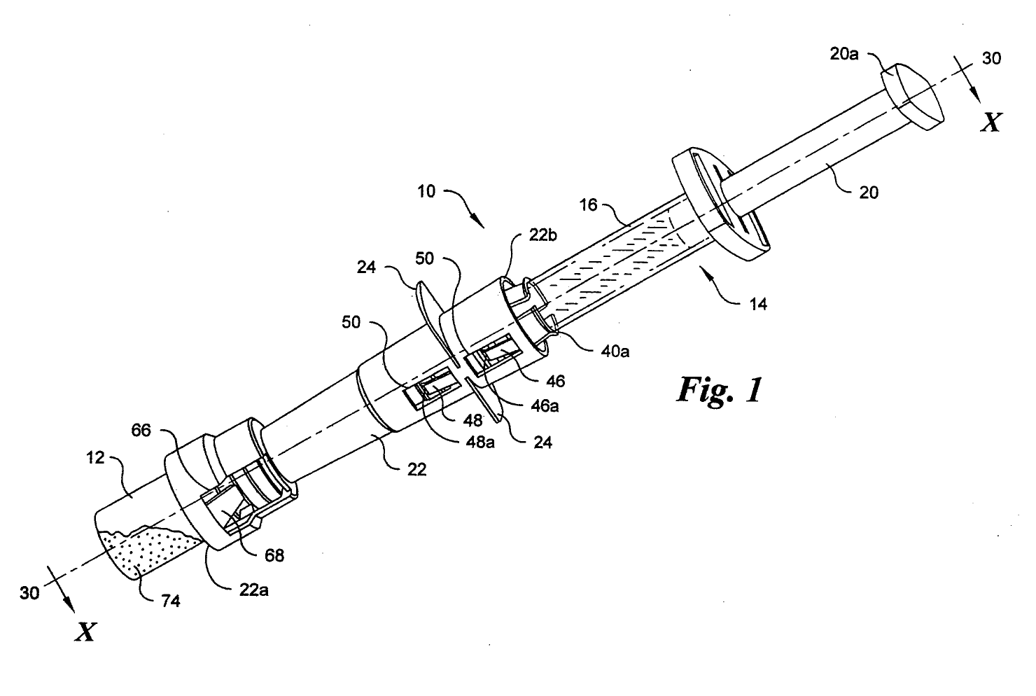

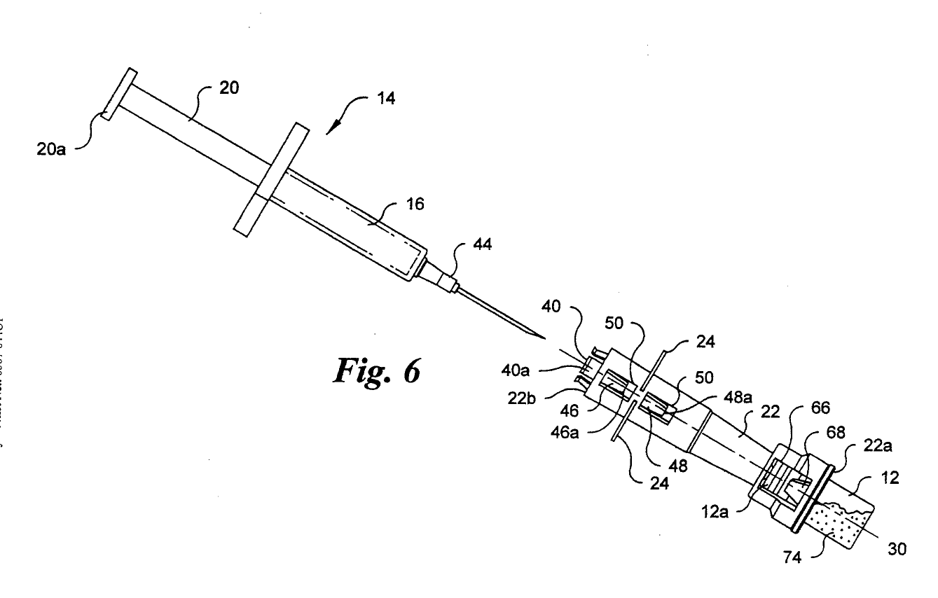

Referring to the drawings in detail, wherein like numerals indicate like elements throughout, there is shown in FIGS. 1-8 a first preferred embodiment of a syringe safety device, generally designated 10, configured to form a fluid coupling between a sealed vial 12 and a syringe 14. The syringe 14 includes a barrel 16 for receiving fluid and a luer cone 18 extending from the barrel 16 for dispensing or receiving fluid. The luer cone 18 has a distal end 18a with a syringe orifice 18b therethrough.

The structure, configuration and operation of the syringe 14 and vial 12 are generally well known by those having ordinary skill in the art. The syringe 14 and vial 12 are typically constructed of a glass material or a transparent or translucent, generally rigid polymeric material that is generally inert or does not react with medications, diluents or other materials that are introduced into the syringe 14 and vial 12. The structure, configuration and operation of the syringe 14 and vial 12 are not described in detail below, as being well known to those having ordinary skill in the art.

Referring to FIGS. 1, 2, 3-3B and 6, the syringe safety device 10 includes a connector 22 having a vial end 22a and a syringe end 22b. The syringe 14 is removably mountable to the syringe end 22b and the vial 12 is mountable to the vial end 22a. The connector 22 preferably has a stepped, cylindrical shape. The vial end 22 preferably has a circular cross-sectional shape that is large enough to at least partially accept the vial 12 therein or, specifically, a peripheral rim 12b of the vial 12, and the syringe end 22b preferably has a circular cross-sectional shape that is large enough to at least partially accept the syringe 14 therein. The connector 22 is preferably constructed of an injection molded, polymeric material and includes a pair of generally rigid grip wings 24 extending generally perpendicularly from an outer surface that are integral with the connector 22. The connector 22 is not limited to injection molded polymeric materials or to the stepped, generally cylindrical configuration shown in the drawings. The connector 22 may have nearly any shape, be processed using nearly any method and be constructed of nearly any material that results in a connector 22 that is able to perform the typical functions of the connector 22 and withstand the normal operating conditions of the connector 22, as is described in greater detail below. The generally stepped, cylindrical shape of the connector 22 is preferred for engagement with the typically cylindrical structures of the syringe 14 and vial 12 and to shield a user from access to sharp needle tips that are positioned in the connector 22 during use. In addition, the polymeric injection molded material is preferred for the connector 22 construction due to its ease of manufacture in producing a relatively complicated shape in an economical manner. For example, the connector 22 may be constructed of an injection molded plastic material or other like materials.

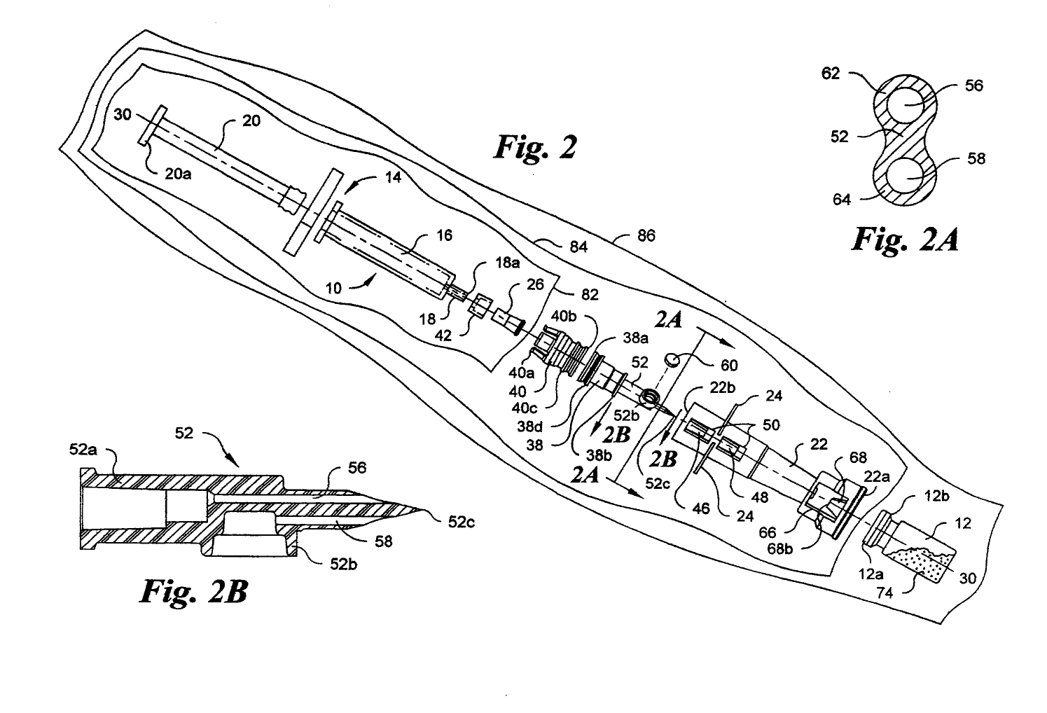

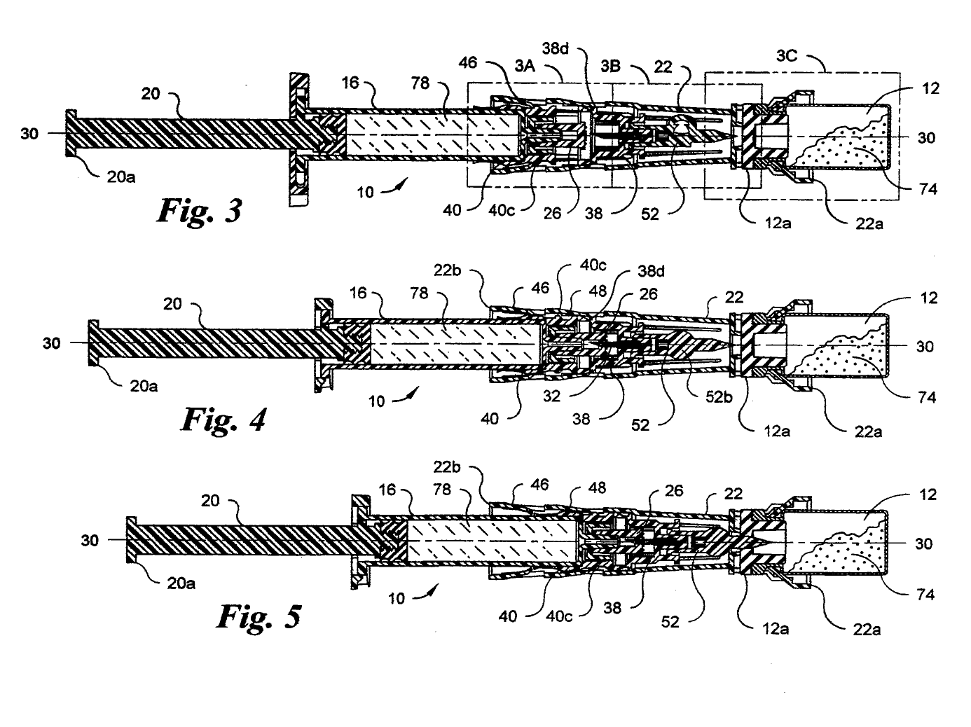

Referring to FIGS. 2, 3A and 7, the syringe safety device 10 also includes a tip cap 26 that is removably mountable to the luer cone 18 of the syringe 14. The tip cap 26 and the distal end 18a of the luer cone 18 define a receiving space 28 in an assembled configuration. In the preferred embodiments, the tip cap 26 is constructed of a self-healing, resilient polymeric material. The tip cap 26 is not limited to self-healing, resilient polymeric materials and may be constructed of nearly any material that is able to take on the general shape, perform the typical functions and withstand the typical operating conditions of the tip cap 26, for example, a thermoplastic elastomer or rubber-like material.

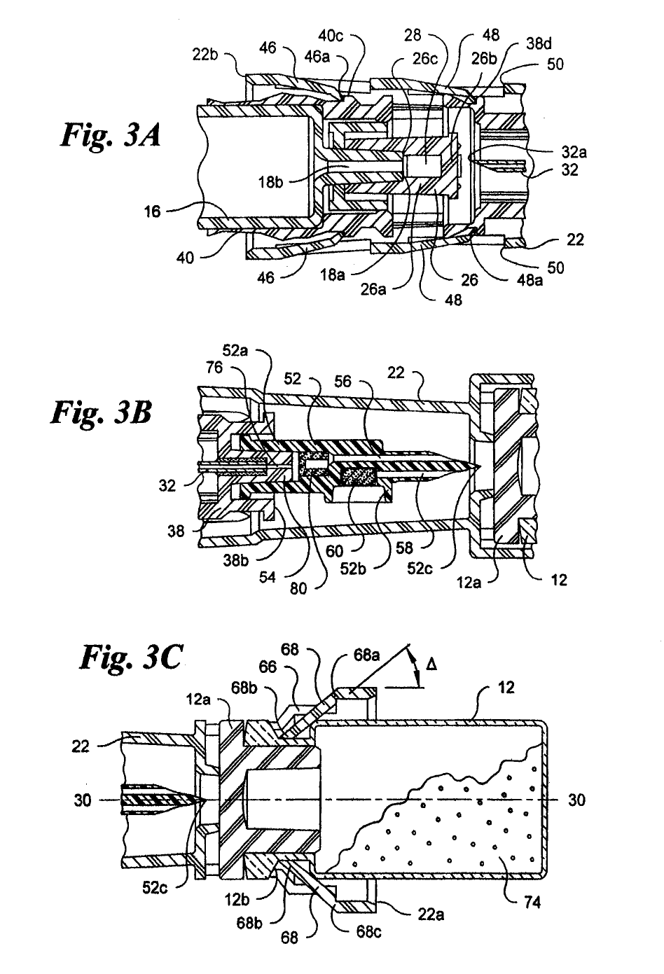

In the preferred embodiments, the tip cap 26 includes a generally cylindrical sleeve 26a and a generally planar penetration wall 26b oriented generally perpendicularly to a connector axis 30 of the syringe safety device 10 in an assembled configuration (FIGS. 3-5 and 7). The penetration wall 26b is preferably positioned at one end of the cylindrical sleeve 26a and a tip mouth 26d is preferably located on an end opposite the cylindrical sleeve 26a. The tip cap 26 is not limited to having a generally cylindrical configuration nor to having the penetration wall 26b positioned at one of its ends. For example, the tip cap 26 may have a generally square or rectangular cross-section and the penetration wall 26b may be positioned somewhere between the two ends of the sleeve 26a, depending upon the configuration of the syringe 14, the luer cone 18 of the syringe 14 and the particular application. The tip cap 26 is generally sized and shaped for mounting to the luer cone 18, as will be described in greater detail below.

The preferred tip cap 26 includes a locating shoulder 26c formed on an inner surface of the sleeve 26a. The luer cone 18 preferably has a generally stepped, cylindrical shape and the distal end 18a of the luer cone 18 contacts the locating shoulder 26c to position the tip cap 26 relative to the distal end 18a in the assembled configuration. The preferred tip cap 26 includes an engaging portion 34 between the shoulder 26c and the tip mouth 26d and a receiving portion 36 between the shoulder 26c and penetration wall 26b. The engaging portion 34 preferably has a larger inner diameter than the receiving portion 36 with the locating shoulder 26c transitioning between the larger diameter of the engaging portion 34 and the smaller diameter of the receiving portion 36. When the tip cap 26 is mounted to the luer cone 18, the engaging portion 34 and the shoulder 26c are preferably in mating contact with the luer cone 18. The resilient, flexible properties of the tip cap 26 allow the tip cap 26 to engage the luer cone 18 along the engaging portion 34 such that a generally fluid-tight seal is created between the tip cap 26 and the luer cone 18. Accordingly, fluid may be contained within the barrel 16, syringe orifice 18b and a receiving space 28 formed in the receiving portion 36 without leakage from the syringe 14. Specifically, the tip cap 26 is preferably force fit onto the luer cone 18 of the syringe 14 such that the receiving portion 36 and penetration wall 26b block fluid from flowing out of the barrel 16, syringe orifice 18b and receiving space 28. The tip cap 26 is not limited to inclusion of separate engaging and receiving portions 34, 36 and may include a receiving space 28 having a relatively constant diameter or a receiving portion 36 having an inner diameter that is larger than the inner diameter of the engaging portion 34.

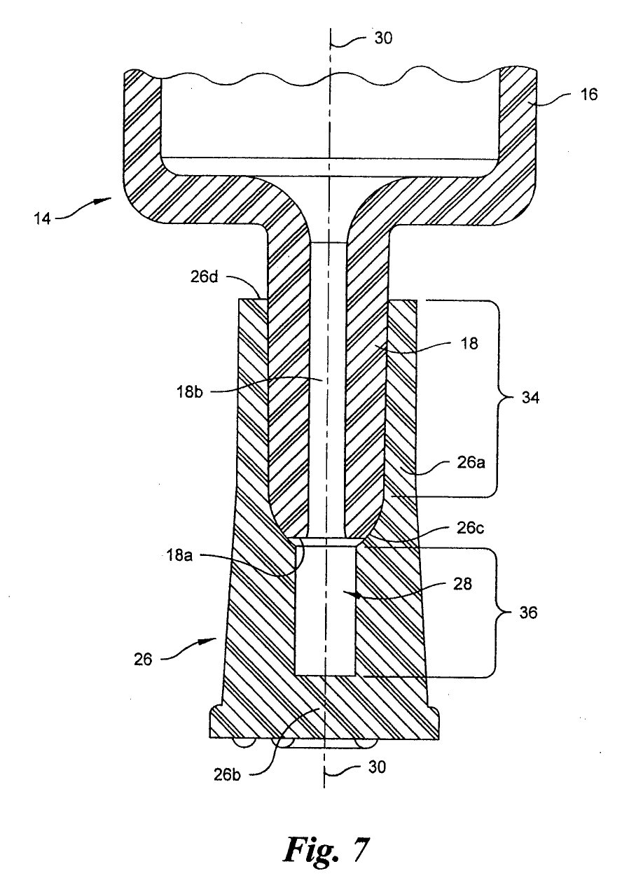

Referring to FIGS. 3-3B and 7, a needle 32 is removably mounted to the connector 22 and includes a needle tip 32a facing toward the syringe 14 in the assembled configuration. The needle tip 32a is positioned in the receiving space 28 when the connector 22 and the syringe 14 are positioned in the engaged position (FIG. 5) or in an intermediate position (FIG. 4) relative to the connector 22. In the first preferred embodiment, the needle 32 does not come into contact with the luer cone 18 in the engaged or intermediate positions. The needle tip 32 is positioned in the receiving space 28 when in the engaged position (FIG. 5) such that the needle 32 does not contact the luer cone 18. Therefore, there is generally no opportunity for the needle tip 32 to potentially become damaged or break by impacting the generally rigid luer cone 18 or damaging the luer cone 18 during an impact. Because the tip cap 26 is generally resilient and constructed of a polymeric material, the needle tip 32a generally easily penetrates the material of the penetration wall 26b of the tip cap 26, thereby generally eliminating any contact between the needle 32 and needle tip 32a and any rigid structure of the syringe safety device 10. This configuration allows the needle tip 32a to contact the fluid in the syringe 14 and create a fluid path out of the receiving space 28, syringe orifice 18b and barrel 16 without ever coming into contact with the rigid luer cone 18 or being positioned in the syringe orifice 18b.

In the first preferred embodiment, the syringe orifice 18b, the receiving space 28 and the needle 32 define at least a portion of a sterile fluid passageway between the syringe 14 and the vial 12 in the engaged position (FIG. 5). Therefore, the elimination of any contact between the needle 32 and the generally rigid luer cone 18 prevents impacts between the needle 32 and luer cone 18 and potential contamination of the fluid within the syringe 14 from chunks of the needle 32 or luer cone 18 that may be dislodged during an impact. In addition, inserting the needle tip 32a into the receiving space 28 as opposed to directly into the syringe orifice 18b in the engaged position permits the use of variable sized needles without concern for matching the diameter of the needle 32 to the inner diameter of the syringe orifice 18b or ensuring that the needle 32 will fit into the syringe orifice 18b regardless of its shape, without interference.

Referring to FIGS. 2, 3, 3B, 4 and 5, in the first preferred embodiment, a sliding linkage 38 is movably mounted within the connector 22 and is movable or slidable along the connector axis 30 therein. The needle 32 is preferably mounted to the sliding linkage 38. The sliding linkage 38 preferably has a generally stepped, cylindrical configuration with a needle end 38a and a spike end 38b but is not so limited. The preferred sliding linkage 38 is constructed of an injection molded, polymeric material. A linkage orifice 76 extends through the sliding linkage 38. The needle 32 is preferably adhesively bonded to the sliding linkage 38 such that a flow path is created between the needle 32 and the linkage orifice 76. The needle 32 is not limited to being adhesively bonded to the sliding linkage 38 and may be mounted to the linkage in nearly any manner including clamping, fastening or through a luer-type lock arrangement as long as the needle 32 creates a fluid path with the linkage orifice 76. In addition, the sliding linkage 38 is not limited to injection molded polymeric constructions and may be constructed of nearly any material able to take on the general shape of the sliding linkage 38 and perform the typical functions of the sliding linkage 38, as will be described in greater detail below.

Referring to FIGS. 1, 2, 3, 3A and 6, in the preferred embodiments, a sliding piston 40 is movably or slidably mounted within the connector 22 along the connector axis 30. The sliding piston 40 has a generally stepped, cylindrical-shape with a first end 40a and a second end 40b. The sliding piston 40 engages the syringe 14 at the first end 40a in the assembled configuration.

The sliding piston 40 is preferably constructed of an injection molded polymeric material and is preferably removably mountable to the first end 40a in the assembled configuration, as will be described in greater detail below. The sliding piston 40 is not limited to stepped, cylindrical shapes or to constructions comprised of injection molded polymeric materials. The sliding piston 40 preferably has a shape that is complementary to sliding engagement with the connector 22 and for removably engaging the syringe 14. The sliding piston 40 may have nearly any shape and may be constructed of nearly any material that is able to perform the typical functions of the sliding joint 40 and withstand the normal operating conditions of the sliding joint 40.

A syringe luer lock 42 is releasably mounted to the luer cone 18 of the syringe 14 in the preferred embodiments. The syringe luer lock 42 is preferably constructed of a generally resilient, polymeric material that is injection molded to form its general shape. One having ordinary skill in the art will realize that the syringe luer lock 42 is not necessary for the operation of the syringe safety device 10 and may be eliminated without significantly impacting the operation of the syringe safety device 10. However, the syringe luer lock 42 is preferred for mounting a luer needle 44 to the luer cone 18 of the syringe 14, as will be described in greater detail below.

Referring to FIG. 7, the penetration wall 26b, cylindrical sleeve 26a and luer cone 18 define the receiving space 28 within which the needle tip 32a is positionable. The sliding piston 40, which is mounted to the barrel 16, may aid in aligning the needle 32 and luer cone 18 such that the needle tip 32a is located in the receiving space 28 in the intermediate or engaged positions. Positioning the needle tip 32a in the receiving space 28 is preferred because insufficient engagement of the needle 32 with the receiving space 28 prevents the creation of a fluid path between the needle 32 and receiving space 28. In contrast, if the needle tip 32a penetrates the penetration wall 26b and moves along or generally parallel to the connector axis 30 beyond the receiving space 28, there is a potential that the needle tip 32a may come into contact with and impact the luer cone 18.

In the preferred embodiments, the tip cap 26 is constructed of a self-healing, polymeric material, for example, a thermoplastic elastomer or rubber-like material. The tip cap 26 is not limited to self-healing, polymeric materials; however, the self-healing material is preferred for construction of at least the penetration wall 26b. The self-healing material in the penetration wall 26b allows the penetration wall 26b to seal-heal after being penetrated by the needle 32 and removing the needle 32 therefrom. Accordingly, even after the needle 32 is removed from the penetration wall 26b, the fluid within the receiving space 28 does not leak from a hole in the penetration wall 26b because the penetration wall 26b self-heals. Self-healing materials are generally well known to those having ordinary skill in the art.

Referring to FIGS. 2, 3 and 7, the cylindrical sleeve 26a includes the tip mouth 26d, which preferably contacts the syringe luer lock 42 in the assembled configuration. Contact between the tip mouth 26d and the syringe luer lock 42 further positions and aligns the tip cap 26 relative to the distal end 18a of the luer cone 18. That is, in addition to locating the tip cap 26 relative to the luer cone 18 using the shoulder 26c, the syringe luer lock 42 contacts the tip mouth 26d such that the tip cap 26 is engaged with the luer cone 18 at a preferred position. The location of the tip cap 26 relative to the distal end 18a of the luer cone 18 aids in defining the receiving space 28 such that the needle tip 32a may be positioned in the receiving space 28 in the intermediate and engaged positions.

In the preferred embodiments, an inner diameter of the cylindrical sleeve 26a within the engaging portion 34 tapers away from the connector axis 30 from the shoulder 26c to the tip mouth 26d. Tapering of the inner surface of the tip cap 26 in the engaging portion 34 in this manner aids in aligning the luer cone 18 relative to the tip cap 26 and promotes the engagement of the tip cap 26 with the luer cone 18 as will be obvious to one having ordinary skill in the art. The tip cap 26 is not limited to being tapered on the inner surface of the engaging portion 34 in this manner and may have a generally constant cross-section in the engaging portion 34 or nearly any cross-section that permits the tip cap 26 to engage the luer cone 18 in a generally fluid-tight manner. The preferred configuration of the tip cap 26 in the engaging portion 34, the configuration and location of the shoulder 26c and the generally resilient nature of the preferred construction of the tip cap 26 allow the tip cap 26 to be force fit onto the luer cone 18 over the engaging portion 34.

Referring to FIGS. 1 and 2, the syringe safety device 10 is utilized for transferring or mixing medications between the syringe 14 and the vial 12. In the engaged position, the needle 32 penetrates the tip cap 26, forming at least a portion of a fluid passageway between the syringe 14 and vial 12. The tip cap 26 is retained on the luer cone 18 when fluid in the vial 12 has been transferred through the fluid passageway into the syringe 14 and the syringe 14 is removed from the syringe end 22b. Accordingly, fluid or a medication that is drawn into the barrel 16 is retained in the barrel 16 by the self-healing tip cap 26. When the syringe 14 is removed from the syringe end 22b, the fluid is not only retained therein, but the tip cap 26 shields the retained fluid from potential external contamination through the exposed syringe orifice 18b, if the tip cap 26 were not positioned on the luer cone 18.

Referring to FIGS. 2, 3A and 6, in the preferred embodiments, a luer needle 44 is removably mountable to the distal end 18a of the syringe 14 when the tip cap 26 is removed from the distal end 18a. When the luer needle 44 is mounted to the luer cone 18, the syringe 14 is prepared for injecting the retained fluid or medication in the barrel 16 into a patient through the luer needle 44. The luer needle 44 is preferably mounted to the luer cone 18 through engagement with the syringe luer lock 42. The syringe luer lock 42 may provide a threaded engagement for mounting to the luer needle 44 or may be shaped such that the luer needle 44 is force fit onto the luer lock 42 and luer cone 18. One having ordinary skill in the art will realize that the luer lock 42 may provide nearly any fastening mechanism to secure the luer needle 44 to the syringe 14 such that the luer needle 44 is in fluid communication with the syringe orifice 18b and is retained on the luer cone 18 during normal operation of the syringe 14. For example, the luer needle 44 may be comprised of a luer slip syringe with no luer threads or a luer lock syringe having threads.

Referring to FIGS. 2 and 3A, the syringe luer lock 42 is removably mountable to the luer cone 18 and may aid in aligning the syringe orifice 18b relative to the sliding piston 40 and the tip cap 26 relative to the syringe orifice 18b in the assembled configuration. Alignment of the sliding piston 40 with the tip cap 26 and the sliding linkage 38 having the needle 32 attached thereto along the connector access 30 in the assembled configuration promotes the proper alignment of the needle 32 relative to the luer cone 18 and placement of the needle tip 32a in receiving space 28 in the intermediate and engaged positions.

Referring to FIGS. 2, 3-3B, 4 and 5, the sliding linkage 38 is movably or slidably mounted to the connector 22 along the connector axis 30. The sliding linkage 38 includes a shoulder 38d adjacent the needle end 38a and the connector 22 includes a first locking mechanism 46 and a second locking mechanism 48 in the preferred embodiments. The sliding piston 40 also includes a shoulder 40c adjacent the second end 40b in the preferred embodiments. The shoulders 38d, 40c of the sliding linkage 38 and sliding piston 40 are preferably integral parts of the sliding linkage 38 and sliding piston 40 formed on an outer surface thereof. The shoulders 38d, 40c are preferably constructed of rings that extend outwardly from the generally cylindrical outer surface of the sliding linkage 38 and sliding piston 40.

The first and second locking mechanisms 46, 48 preferably permit the sliding linkage 38 and sliding piston 40 to move in an engaging direction along the connector axis 22 toward the vial end 22a and preferably prevent the sliding linkage 38 and sliding piston 40 from moving in a release direction opposite the engaging direction beyond a first locking position (FIG. 1) and a second locking position (FIG. 3) once the sliding linkage 38 and sliding piston 40 have moved in the engaging direction past the first and second locking positions, respectively. That is, the sliding linkage 38 and sliding piston 40 are able to slide in the engaging direction toward the vial end 22a such that the shoulders 38d, 40c move to and beyond the first and second locking mechanisms 46, 48 and pass the first and second locking positions, but are preferably unable to travel in the disengaging direction beyond the first and second locking positions once they have traveled to and beyond the first and second locking positions. The sliding linkage 38 and sliding piston 40 are preferably configured in this manner such that they are retained in the connector 22 once they are mounted in the assembled configuration and do not fall out of the connector 22 during use. However, the sliding linkage 38 and sliding piston 40 are not so limited and may be releaseable from the connector 22 for subsequent use, as will be understood by one having ordinary skill in the art. In the preferred embodiments, an audible indication or notice sounds when the shoulders 38d, 40c slide along the connector axis 30 in the engaging direction beyond the first and second locking mechanisms 46, 48, as will be described in greater detail below.

The sliding linkage 38 and sliding piston 40 are guided in their movement along the connector axis 30 by contact between the shoulders 38d, 40c or other portions of the sliding linkage 38 and sliding piston 40 and an inner surface of the connector 22. The generally cylindrical shape of the sliding linkage 38 and sliding piston 40 and the generally hollow, cylindrical shape of the connector 22 align the sliding linkage 38 and sliding piston 40 along the connector axis 30 and permit movement of the sliding linkage 38 and sliding piston 40 along the connector axis 30. However, the sliding linkage 38 and sliding piston 40 are not limited to being slidably mounted to the connector 22 in the above-described manner and may, for example, mount on rails (not shown) in the connector 22 or include ball bearings (not shown) that permit slidable mounting of the sliding linkage 38 and sliding piston 40 within the connector 22.

Referring to FIGS. 1, 2, 3, 3A and 4-6, in the preferred embodiments, the first and second locking mechanisms 46, 48 are comprised of a first and second pair of resilient tabs 46, 48 that are positioned in cutouts 50 in the connector 22. The resilient tabs 46, 48 extend inwardly from the connector 22 toward the connector axis 30 and include grasping tips 46a, 48a that are engageable with the shoulders 38d, 40c. The tabs 46, 48 are flexible in that the grasping tips 46a, 48a are able to flex away from or toward the connector axis 30 when a force is applied to the tips 46a, 48a generally radially away from the connection axis 36. When the sliding linkage 38 or sliding piston 40 is urged into the connector 22 in the engaging direction, the shoulders 38d, 40c urge the tips 46a, 48a away from the connector axis 30 to allow the sliding linkage 38 or sliding piston 40 to move toward the vial end 22a. When the shoulders 38d, 40c move beyond the grasping tips 46a, 48a in the engaging direction, the shape of the tabs 46, 48 and shoulders 38d, 40c generally prevents the sliding linkage 38 or sliding piston 40 from moving in the disengaging direction toward the syringe end 22b. In the initial position (FIG. 3) the sliding linkage 38 is engaged with the second locking mechanism 48 at its shoulder 38d and the sliding piston 40 is engaged with the first locking mechanism 46 at its shoulder 40c. Accordingly, in the initial position, the sliding linkage 38 and sliding piston 40 are generally prevented from moving in the disengaging direction toward the syringe end 22 beyond the first locking mechanism 46 or second locking mechanism 48, respectively. Therefore, the sliding linkage 38 and sliding piston 40 are preferably secured in the connector 22 in the initial position.

One having ordinary skill in the art will realize that the sliding linkage 38 and sliding piston 40 are movable in the disengaging direction beyond the first and second locking mechanisms 46, 48 by bending the tabs 46, 48 outwardly to allow the shoulders 38d, 40c to pass beyond the grasping tips 46a, 48a. In addition, one having ordinary skill in the art will realize that the connector 22 is not limited to the specific tabs 46, 48 having grasping tips 46a, 48a shown in the figures and may include nearly any locking mechanism that allows movement of the sliding linkage 38 and sliding piston 40 in the engaging direction along the connector axis 30 and generally prevents movement of the sliding linkage 38 and sliding piston 40 in the disengaging direction once the sliding linkage 38 and sliding piston 40 have moved beyond the first or second locking mechanisms 46, 48 in the engaging direction.

Referring to FIGS. 1, 2 and 3A in the preferred embodiments, the syringe 14 is releasably engageable by the sliding piston 40 adjacent the luer cone 18 in the assembled configuration. The sliding piston 40 preferably aligns the syringe 14 relative to the connector 22 along the connector axis 30. In the assembled configuration, when the sliding piston 40, with the syringe 14 attached thereto, slides in the engaging direction such that the shoulder 40c is engaged by the first locking mechanism 46, the syringe 14 may be released from the sliding piston 40 by moving the syringe 14 in the disengaging direction. The sliding piston 40 is preferably retained in the connector 22 through engagement of the first grasping tip 46a with the shoulder 40c. Accordingly, although the sliding piston 40 is generally not removed from the connector 20 by applying a force in the disengaging direction once the shoulder 40c is engaged with the grasping tip 46a, the syringe 14 may be released from the sliding piston 40 by applying a force in the disengaging direction. The tip cap 26 is preferably retained on the luer cone 18 when the syringe 14 is removed from the connector 22 and releases from the sliding piston 40, but is not so limited. For example, the tip cap 26 may be retained in the connector 22 upon removal of the syringe 14 from the connector 22.

Referring to FIGS. 1, 2 and 3B, the connector 22 receives the sealed vial 12 and syringe 14 at the vial end 22a and syringe end 22b, respectively, in the assembled configuration. The vial 12, connector 22 and syringe 14 are preferably aligned along the connector axis 30 in the assembled configuration. In addition, the sliding linkage 38 and sliding piston 40 are mounted within the connector 22 along the connector axis 30 and are slidable along the connector axis 30, typically in the engaging direction. The needle 32 is mounted to the sliding linkage 38 adjacent the needle end 38a and a spike 52 is mounted to the sliding linkage 38 adjacent the spike end 38b. The needle 32 and spike 52 are positioned generally coaxially on the connector axis 30 in the assembled configuration, but are not so limited. For example, the needle 32 and spike 52 may be aligned generally parallel to the connector axis 30. In the first preferred embodiment, the spike 52 includes a first or female luer lock connector 52a that releasably mounts to a second or male luer lock connector 54 of the sliding linkage 38.

One having ordinary skill in the art will realize that the spike 52 is not limited to being luer locked to the sliding linkage 38. For example, the spike 52 may be adhesively bonded to the sliding linkage 38. Luer-locking the spike 52 to the sliding linkage 38 allows the spike 52 to be removed from the sliding linkage 38, cleaned and potentially reused in an additional operation.

Referring to FIGS. 3A-3C and 5, in the first preferred embodiment, the spike 52 pierces the vial stopper 12a and the needle 32 pierces the tip cap 26 on the distal end 18a of the syringe 14 in the engaged position (FIG. 5). Accordingly, when the needle 32 pierces the tip cap 26 and the spike 52 pierces the vial stopper 12a, a sterile fluid passageway is created between the vial 12 and syringe 14 through the needle 32, a fluid lumen 56 in the spike 52 and the linkage orifice 76 of the sliding linkage 38. Fluids, solutions or gases may be passed to and between the syringe 14 and vial 12 through the sterile fluid passageway.

Referring to FIGS. 2-2B, 3B and 5, in the first preferred embodiment, the spike 52 includes an air filter housing 52b that is preferably integrally formed with the spike 52. In addition, the spike 52 includes the fluid lumen or first channel 56 and a second channel 58. The first channel 56 extends from a location proximate an end of the spike 52 adjacent the female luer lock connector 52a to a position proximate a penetration end 52c of the spike 52 and the second channel 58 extends from the air filter housing 52b to a position proximate the penetration end 52c. The first and second channels 56, 58 are separate and are both preferably exposed to an internal cavity of the vial 12 beneath the vial stopper 12a in the engaged position. The first channel 56 preferably forms part of the sterile fluid passageway between the syringe 14 and the vial 12 in the engaged position. The second channel 58 preferably forms a fluid passageway between the vial 12 and an external environment through the filter housing 52b. The first and second channels 56, 58 preferably extend generally parallel relative to each other and are generally parallel to the connection axis 30 in the assembled configuration.

The spike 52 is not limited to the inclusion of the second channel 58 or to the inclusion of the air filter housing 52b. For example, the spike 52 may be comprised of two needles (not shown) that are mounted to the sliding linkage 38, one that forms part of the fluid passageway between the vial 12 and the syringe 14 in the engaged position and one that provides a passageway to the external environment to relieve pressure in the vial 12 during use, as will be described in greater detail below. In addition, the spike 52 may be formed with a single passageway (not shown) that forms part of the fluid passageway. This configuration is preferably utilized with vials 12 and syringes 14 having a relatively small volume where balancing the pressure inside of the vial 12 with atmosphere is often unnecessary. One having ordinary skill in the art will realize that the spike 52 may be comprised of nearly any mechanism that provides a portion of a flow path between the vial 12 and syringe 14, is able to penetrate the vial stopper 12a and slide along the connector axis 30 with the sliding linkage 38.

In the first preferred embodiment, an air filter 60 is mounted within the air filter housing 52b of the spike 52. The air filter 60 preferably filters air passing to and from the vial 12 from the external environment through the second channel 58. When fluid from the syringe 14 is introduced into the vial 12 through the sterile fluid passageway, air within the vial 12 is preferably forced out of the vial 12 by the incoming fluid through the second channel 58 and through the air filter 60. Conversely, when fluid is drawn out of the vial 12 through the sterile fluid passageway and into the syringe 14, air is drawn into the vial 12 through the air filter 60 and the second channel 58. Drawing fluid out of the vial 12 typically creates a vacuum that draws air through the air filter 60, through the second channel 58 and into the vial 12. The air filter 60 is preferably able to filter external air that may potentially include contaminants that could contaminate the fluid or medication within the syringe 14 or the vial 12. In addition, the inclusion of the second channel 58, which provides exposure to the external environment in the engaged position, aids in stabilizing pressure within the vial 12, which is preferred for relatively large vials 12 or the dispensing or introduction of large amounts of fluid or medication into or out of the vial 12. The introduction of relatively large volumes of fluids into a relatively large vial 12 often creates a high pressure in the vial 12 and may damage the vial 12 or push the vial stopper 12a out of the vial 12 during use, thereby potentially spilling the fluid and/or medication. One having ordinary skill in the art will realize that the syringe safety device 10 is not limited to the inclusion of the air filter 60 or air filter housing 52b and may relieve pressure or balance the vacuum within the vial 12 simply using the second channel 58. In addition, one having ordinary skill in the art will realize that the inclusion of the second channel 58 is not required for the operation of the syringe safety device 10 and is provided generally for relatively large vials 12, which may be subject to relatively large pressure or vacuum variations during use.

Referring to FIGS. 2-2B and 3B, the penetration end 52c of the spike 52 faces the vial end 22a in the assembled configuration for piercing the vial stopper 12a in the engaged position. The penetration end 52c has a generally figure eight-shaped cross-sectional configuration (FIG. 2A). The spike 52 includes a first bulbous region 62 and a second bulbous region 64 adjacent the penetration end 52c. The first channel or fluid lumen 56 is preferably positioned in the first bulbous region 62 and the second channel or air bleed lumen 58 is preferably positioned in the second bulbous region 64. The figure eight-shaped cross-sectional configuration of the penetration end 52c of the spike 52 limits the cross-sectional area of the penetration end 52c while accommodating both the fluid lumen 56 and the air bleed lumen 58. Construction of the spike 52 having a conventional circular cross-section at its penetration end 52c results in a relatively large cross-sectional area penetrating the vial stopper 12a. In contrast, utilization of the figure eight-shaped cross-section results in a smaller cross-sectional area that penetrates the vial stopper 12a. Accordingly, a hole in the vial stopper 12a is comparatively smaller, the force required to penetrate the vial stopper 12a with the spike 52 is comparatively smaller and the resulting hole in the vial stopper 12a that must be self-healed by the vial stopper material is comparatively smaller. In addition, the figure eight-shaped cross-sectional configuration is able to accommodate both the fluid and air bleed lumens 56, 58 for introduction into the vial 12 in the engaged position.

One having ordinary skill in the art will realize that the figure eight-shaped, cross-sectional configuration of the penetration end 52c of the spike 52 is not limiting and the syringe safety device 10 may operate having a spike 52 with the fluid and air bleed lumens 56, 58 therein and a generally circular cross-section or with a spike 52 having a completely different configuration, as was described above. However, the figure eight-shaped, cross-sectional configuration is preferred for the above-stated reasons.

Referring to FIGS. 1, 2, 3C, 6 and 8, the connector 22 includes a lock window 66 adjacent the vial end 22a. A vial lock 68 extends toward the connector axis 30 into the lock window 66 from a root end 68a adjacent the vial end 22a to a terminal end 68b. The terminal end 68b is in engagement with the peripheral rim 12d when the vial 12 is mounted to the vial end 22a. The lock window 66 is preferably a generally rectangular cutout in the connector 22 adjacent the vial end 22a and the vial lock 68 preferably extends from an edge of the lock window 66 adjacent the vial end 22a to the terminal end 68b toward the connector axis 30. The vial lock 68 is preferably comprised of a pair of vial lock tabs 68 that are integrally formed with the connector 22. The vial lock tabs 68 are preferably resilient and may flex at their terminal end 68b toward and away from the connector axis 30. In addition, the lock window 66 is preferably comprised of first and second windows 68 that incorporate the vial locks 68 on opposing sides of the connector 22.

In the preferred embodiments, the connector 22 includes a vial shoulder 70 that is positioned on a plane generally perpendicular relative to the connector axis 30 in the assembled configuration. The vial shoulder 70 includes a spike hole 72 therein that is generally centered about the connector axis 30. In the engaged position, the vial stopper 12a is preferably in facing engagement with the vial shoulder 70. The vial shoulder 70 aids in positioning the vial 12 relative to the connector 22 when the vial 12 is mounted thereon. One having ordinary skill in the art will realize that the connector 22 is not limited to the inclusion of the vial shoulder 70. The vial shoulder 70 is preferably provided to aid in positioning the vial 12 relative to the connector 22 and to limit access to the penetration end 52c of the spike 52. In addition, the vial shoulder 70 aids in engaging or mounting the vial 12 to the connector 22 with the vial locks 68, as will be described in greater detail below.

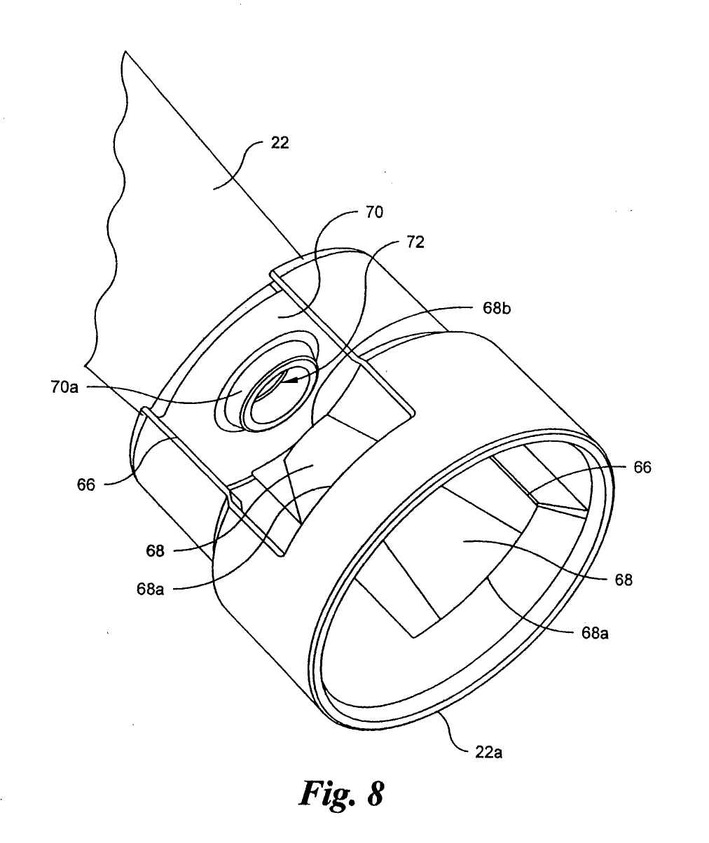

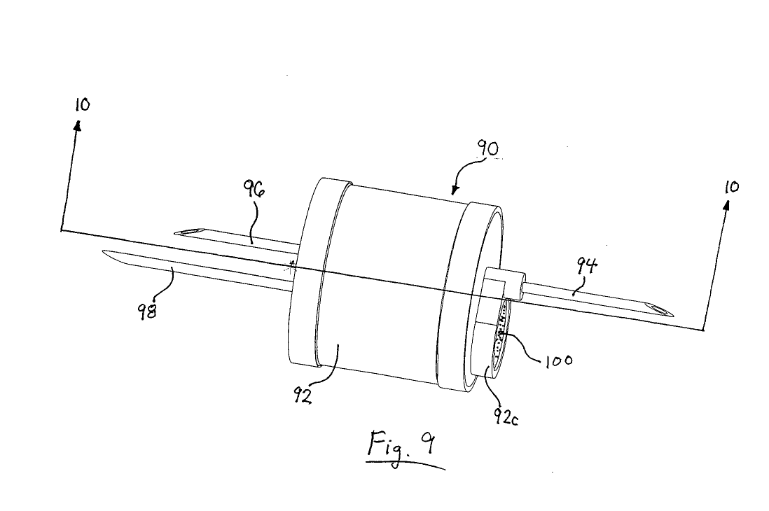

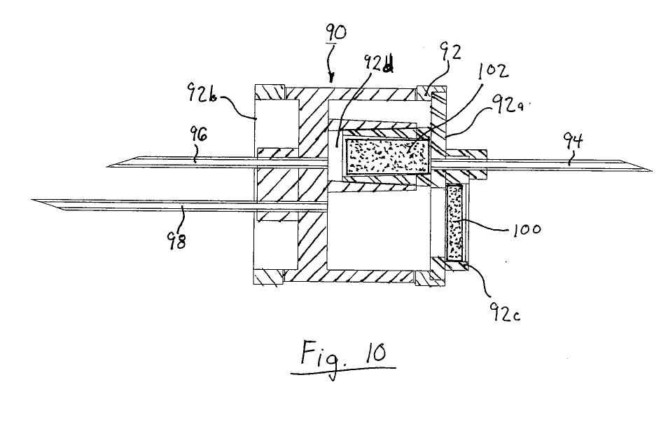

Referring to FIG. 8, in the preferred embodiments, the vial shoulder 70 includes a ring 70a that defines the spike hole 72. The ring 70a is preferably in facing engagement with the vial stopper 12a when the vial 12 is mounted to the connector 22. The vial ring 70a is preferably provided for alignment and strength purposes and is not necessarily required for the operation of the syringe safety device 10, as will be understood by one having ordinary skill in the art.

Referring to FIG. 3C, the vial lock 68 preferably extends from the root end 68a to the terminal end 68b at a lock angle Δ relative to the connector axis 30. In the first preferred embodiment, the lock angle Δ is between thirty and fifty degrees (30°-50°). The preferred vial lock 68 is resilient to permit flexure of the vial lock 68 toward and away from the connector axis 30. Accordingly, to mount the vial 12 to the connector 22 in the first preferred embodiment, the vial 12 is urged along the connector axis 30 in the disengaging direction toward the vial shoulder 70. As the vial stopper 12a nears the vial shoulder 70, the vial lock 68 contacts the vial stopper 12a on opposing sides and flexes outwardly relative to the connector axis 30. As the vial 12 moves closer to the vial shoulder 70, the terminal end 68b of the vial lock 68 clears the peripheral rim 12b of the vial 12 and flexes toward the connector axis 30. In the assembled configuration, the terminal end 68b is in engagement with the peripheral rim 12b. Due to the shape and construction of the vial lock 68 and the peripheral rim 12b, the vial 12 resists being moved away from the vial shoulder 70 once the vial 12 is engaged by the vial lock 68.

In the first preferred embodiment, when the terminal end 68b clears the outer diameter of the peripheral rim 12b, the vial lock 68 snaps inwardly and creates an audible indication or a “click”. The audible indication or “click” indicates that the vial 12 is secured to the connector 22 by the vial lock 68. One having ordinary skill in the art will realize that the audible indication is desirable for providing an indication to the user when the vial 12 is properly engaged with the connector 22. However, the audible indication is not limiting and the syringe safety device 10 will operate without the audible indication. In addition, the specific configuration of the vial lock 68 is not limiting and the vial 12 may be mounted to the connector 22 using nearly any mounting mechanism that secures the vial 12 to the vial end 22a, for example, clamping, mechanical fastening, adhesive bonding or other similar mounting mechanisms. For example, the connector 22 may include a pair of spring-biased tabs (not shown) similar to the vial lock 68 that are pivotally mounted proximate the vial end 22a. The spring biased tabs may be manually pivotable by a user to engage and/or disengage the peripheral rim 12b of the vial 12. The pivotable tabs are preferred for a connector 22 that may be reused several times, such that several different vials 12 may be engaged and disengaged by the connector 22.

In the first preferred embodiment, the vial lock 68 generally prevents the vial 12 from moving in a release direction along the connector axis 30 when the vial 12 is mounted to the vial end 22a without a force being applied to the vial lock 68 radially outwardly relative to the connector axis 30. Due to limited access to an inner side of the vial lock 68, an outward force is relatively difficult to apply to the vial lock 68 and the vial 12 is generally secured to the connector 22 once the vial lock 68 engages the peripheral rim 12b. Securing the vial 12 to the connector 22 and preventing release of the vial 12 from the connector 22 is desirable to prevent the vial 12 from disengaging from the connector 22 during use. In addition, it is preferred that the vial 12 and connector 22 are disposed of or thrown away after a single use.

Referring to FIGS. 1-6, in operation, medication may be transferred between the syringe 14 and the vial 12 using the syringe safety device 10. The sliding linkage 38 and sliding piston 40 are assembled along the connector axis 30 within the connector 22 in the initial position (FIG. 3). In this position, the shoulder 38d of the sliding linkage 38 is engaged by the second locking mechanism 48 and the shoulder 40c of the sliding piston 40 is engaged by the first locking mechanism 46. The syringe 14 with the tip cap 26 applied to the luer cone 18 is engaged along the connector axis 30 to the first end 40a of the sliding piston 40 and the vial 12 is mounted to the vial end 22a. A force is initially applied to the syringe 14 in the engaging direction, thereby urging the syringe 14 and sliding piston 40 along the connector axis 30 toward the vial 12. This initial movement causes the needle 32 to penetrate the tip cap 26 and position the needle tip 32a in the receiving space 28 in the intermediate position (FIG. 4). Additional force is applied to the syringe 14 in the engaging direction and the sliding piston 40 engages the sliding linkage 38 at the needle end 38a. Additional force is applied to the syringe 14 along the connector axis 30 toward the vial 12, urging the sliding piston 40 and sliding linkage 38 along the connector axis 30 such that the spike 52 penetrates the vial stopper 12a. The sterile fluid passageway is thereby created between the syringe 14 and the vial 12 in the engaged position (FIG. 5). Medication may be drawn from the vial 12 through the sterile fluid passageway into the barrel 16 by drawing the plunger 20 along the connector axis 30 away from the vial 12. The syringe 14 may then be withdrawn from the sliding piston 40 and the connector 22 by applying a force to the syringe 14 away from the vial 12 or in the disengaging direction along the connector axis 30. The sliding piston 40 is preferably retained in the connector 22 through engagement of the shoulder 40c with the second locking mechanism 48. The syringe 14 is released from the connector 22 with the tip cap 26 mounted thereon to retain the medication that is within the barrel 16 and protect the medication from potential contamination. The tip cap 26 may then be removed from the luer cone 18 and the luer needle 44 may be engaged with the luer cone 18 for injecting the medication into a patient.

Alternatively, when the vial 12 and syringe 14 are in the engaged position, a diluent 78 from the syringe 14 may be introduced into the vial 12 through the sterile fluid passageway. The vial 12 may include powdered medication 74 therein for mixing with the diluent 78. The diluent 78 and powdered medication 74 are preferably mixed by agitating the vial 12 while the syringe safety device 10 is in the engaged position. When the diluent 78 and powdered medication 74 are sufficiently mixed, a medication solution results. The medication solution may be drawn into the syringe 14 by drawing the plunger 20 away from the vial 12 in the disengaging direction and drawing the medication solution through the sterile fluid passageway into the barrel 16. The syringe 14 is then removed from the sliding piston 40, the tip cap 26 is removed from the luer cone 18 and the luer needle 44 is engaged with the luer cone 18 for injecting the medication solution into the patient. The syringe safety device 10 may also be configured such that the powdered medication 74 is enclosed in the syringe 14 such that diluent may be introduced into the syringe 14 from the vial 12 for mixing of the medication in the syringe 14.

Referring to FIGS. 1, 2 and 6, after the medication has been transferred from the vial 12 into the syringe 14 or a medication solution has been created and drawn into the syringe 14 and the syringe 14 has been removed from the sliding piston 40 and connector 22, the connector 22, sliding piston 40, sliding linkage 38 and vial 12 as well as their related components may be disposed of. The sliding linkage 38, sliding piston 40, connector 22 and vial 12 are preferably disposable after a single use. Cleaning and/or sterilizing of these components after a single use is not preferred but may be performed and the components may be used numerous times.

Referring to FIGS. 1, 2, 3, 3A and 4, in the preferred embodiments, when the sliding piston 40 slides into the connector 22 and is mounted in the initial position, an audible indication or “click” sounds informing the user that the sliding piston 40 is engaged by the connector 22. One having ordinary skill in the art will realize that the audible indication is not required for proper operation of the syringe safety device 10 but is preferable to provide an indication to the user that the sliding piston 40 is engaged in the initial position by the connector 22. The audible indication is preferably provided by the first locking mechanism 46 clearing the shoulder 46c and flexing inwardly into contact with an outer surface of the sliding piston 40.

Referring to FIGS. 1 and 3A-5, in the first preferred embodiment, when the penetration end 52c of the spike 52 is moved from the intermediate position to the engaged position, an audible indication or “click” sounds informing the user that the spike 52 and penetration end 52a are positioned in the vial 12 beneath the vial stopper 12a. In the engaged position, a fluid passageway is preferably formed between the vial 12 and syringe 14 through the syringe safety device 10. One having ordinary skill in the art will realize that the audible indication is not required for proper operation of the syringe safety device 10 but is preferable to provide an indication to the user that the movement of the syringe 14 toward the vial 12 and subsequent penetration of the vial stopper 12a by the spike 52 is progressing in a normal manner between the intermediate position and the engaged position. The audible indication is preferably provided by the second locking mechanism 48 clearing the shoulder 46c and flexing inwardly into contact with an outer surface of the sliding piston 40. The sliding linkage 38 is urged from the intermediate position to the engaged position through contact with the sliding piston 40 when force is applied to the syringe 14 toward the vial 12.

Referring to FIGS. 1, 3, 3A, 4, 5 and 7, the syringe safety device 10 is preferably distributed by a manufacturer by at least partially filling the syringe 14 with a diluent 78. The tip cap 26 is then mounted to the luer cone 18 such that the diluent 78 is retained in the barrel 16, the syringe orifice 18b and the receiving space 28. The diluent filled syringe 14 is shipped with the tip cap 26 thereon to a remote location where the filled syringe 14 is assembled with a fluid transfer assembly including the connector 22 with the sliding linkage 38 and sliding piston 40 mounted therein and their associated components. The syringe 14 assembled with the fluid transfer assembly is delivered to an end user. The end user typically mounts the vial 12 with the powdered medication 74 therein onto the vial end 22a of the connector 22 for introduction of the diluent 16 into the vial 12, mixes the medication solution by mixing the diluent 78 with the powdered medication 74, draws the medication solution into the barrel 16, removes the syringe 14 with the tip cap 26 mounted thereon, removes the tip cap 26 from the luer cone 18, mounts the luer needle 44 onto the luer cone 18 and injects the medication solution from the syringe 14 into the patient through the luer needle 44.

The vial 12 is not limited to including the powdered medication 74 therein and the syringe 14 is not limited to including diluent therein. For example, the vial 12 may include diluent therein and the syringe 14 may include the powdered medication 74 therein or the vial 12 may include a concentrated, liquid-form medication therein and the syringe 14 may include a diluted medication therein. The process of transferring materials to and/or from the vial 12 to the syringe 14 with the above-described medications and/or diluents in the vial 12 and syringe 14 using the syringe safety device 10 will be understood by one having ordinary skill in the art.

Referring to FIGS. 1 and 8, in the first preferred embodiment, when the vial 12 is mounted to the vial end 22a of the connector 22, an audible indication or “click” sounds indicating to the user that the vial 12 is locked or mounted to the connector 22. The audible indication or click sound is preferably comprised of the pair of vial locks 68 flexing inwardly and contacting a neck of the vial 12 after the vial locks 68 have cleared the peripheral rim 12b of the vial 12. One having ordinary skill in the art will realize that the audible indication notifying the user that the vial 12 is mounted to the connector 22 is not limiting. The audible indication is preferred to notify the user that the vial 12 is mounted or locked to the vial end 22a of the connector 22 and is prepared for operation.

Referring to FIGS. 1, 3, 4 and 5, in the preferred embodiments, once the syringe safety device 10 is in the engaged position, the diluent 78 is dispensed through the fluid passageway into the vial 12 by depressing the plunger 20 toward the vial 12 along the connector axis 30. Depressing the plunger 20 toward the vial 12 urges the diluent 78 in the barrel 16 through the syringe orifice 18b, into the receiving space 28, through the needle 32, through the linkage orifice 76, through a fluid filter 80 mounted in the spike 52, through the first channel 56 and into the vial 12. Any pressure that may be created in the vial 12 through introduction of the diluent 78 may be relieved by releasing fluid or gases through the second channel 58, through the air filter 60 in the spike 52 and out of the air filter housing 52b. When it is desirable to draw the fluid within the vial 12 back into the syringe 14, the plunger 20 is drawn along the connector axis 30 away from the vial 12, thereby creating a vacuum in the barrel 16 to draw the fluid in the reverse direction along the fluid passageway. Any vacuum that may be created in the vial 12 by drawing the fluid in the vial 12 back into the syringe 14 may be relieved by drawing atmospheric air through the air filter 60, through the second channel 58 and into the vial 12. When a sufficient amount of fluid has been drawn into the barrel 16, the syringe 14 may be removed from the connector 22 and sliding piston 40 by applying a force to the syringe 14 along the connector axis 30 away from the vial 12 in the disengaging direction. The penetration in the tip cap 26 from the needle 32 is self-healed by the material of the tip cap 26 and the fluid is retained within the barrel 26, syringe orifice 18b and receiving space 28.

Referring to FIG. 2, in the preferred embodiments, when the syringe 14 with the tip cap 26 mounted thereon is shipped to a remote location, as was described above, the syringe 14 and tip cap 26 assembly is packaged in a first sterile package 82. Prior to assembling the syringe 14 with the tip cap 26 assembled thereto to the connector 22 and the remainder of the fluid transfer assembly, the syringe 14 and tip cap 26 assembly is removed from the first sterile package 82. The first sterile package 82 is preferred to maintain the sterility of the syringe 14, syringe luer lock 42 and tip cap 26 prior to use. One having ordinary skill in the art will realize that the first sterile package 82 is not required for the shipment of the syringe 14 and tip cap 26 assembly. Alternatively, a plurality of syringe 14 and tip cap 26 assemblies may be transported in a single first sterile package 82 as opposed to being individually packaged or may not be shipped in a sterile package at all. It is preferred that the syringe 14 and tip cap 26 assembly remain in their sterile state prior to use to avoid any potential contamination of the diluent 78 or any medication in the barrel 16 that may come into contact with the barrel 16 or tip cap 26 prior to use.

In the preferred embodiments, when the syringe 14 and tip cap 26 assembly is mounted to the fluid transfer assembly, the overall assembly is packaged in a second sterile package 84 prior to delivering the assembly to an end user. The entire assembly is preferably packaged in the second sterile package 84 to generally prevent contamination of any of the components of the assembly during shipping. The end user is then able to remove the total assembly from the second sterile package 84 prior to use. Specifically, the end user typically removes the assembly from the second sterile package 84 prior to mounting the vial 12 to the vial end 22a of the connector 22. One having ordinary skill in the art will realize that the syringe 14 with the tip cap 26 mounted thereon and assembled to the connector 22 is not limited to being packaged in the second sterile package 84 before shipment to an end user.

The assembled syringe safety device 10 may be packaged in a third sterile package 86 for shipment directly to an end user. The syringe safety device 10 may be packaged in the third sterile package 86 in the initial position (FIG. 3) such that the device 10 is prepared for use as soon as it is removed from the third sterile package 86. However, the components of the syringe safety device 10 may also be individually enclosed in the third sterile package 86 for assembly by the user or shipped in alternative configurations.

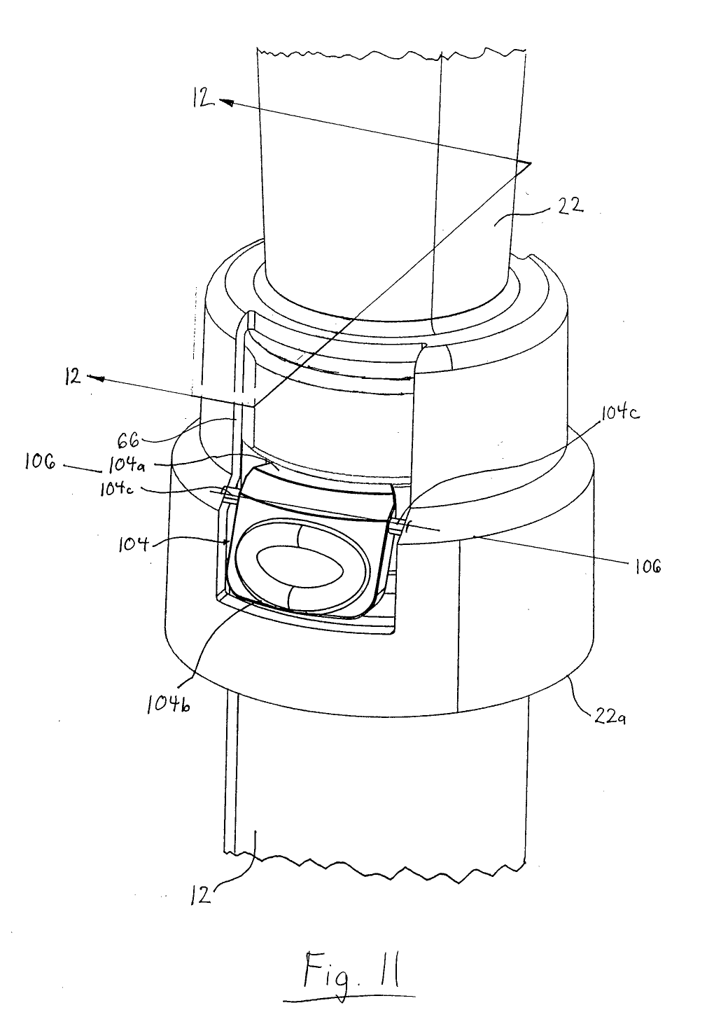

Referring to FIGS. 9 and 10, in a second preferred embodiment, the spike 52 and sliding linkage 38 of the first preferred embodiment may be replaced in the assembly of the syringe safety device 10 by a double needle assembly 90. The double needle assembly 90 preferably includes a double needle housing 92, and a syringe needle 94, a vial needle 96 and a vent needle 98 that are each mounted to the housing 92. The syringe, vial and vent needles 94, 96, 98 are preferably fixed at a blunt end to the double needle housing 92 and extend in a longitudinal direction away from the housing 92 to a generally sharp tip with the syringe needle 94 extending from a first end 92a of the housing 92 and the vial and vent needles 96, 98 extending from a second, opposite end 92b. The housing 92 also includes an air filter housing 92c extending from the first end 92a and a fluid cavity 92d that the blunt ends of the syringe and vial needles 94, 96 open into. An air filter 100 is removably mountable in the air filter housing 92c and a fluid filter 102 is mounted in the fluid cavity 92d. The air filter 100 may also be permanently attached or fixed to the air filter housing 92c. The housing 92 is preferably constructed of an injection molded polymeric material and the needles 94, 96, 98 may be integrally molded into, adhesively bonded or removably secured to the double needle housing 92.

Referring to FIGS. 2 and 3-5, in operation, the double needle housing 92 with the syringe, vial and vent needles 94, 96, 98 mounted thereto and the fluid and air filters 100, 102 mounted therein is slidably inserted into the connector 22 such that the second end 92b is facing the vial end 22a. The external surface of the double needle housing 92 is preferably constructed to mate with the first and second locking mechanisms 46, 48 and an inner diameter of the connector 22 to slidably secure and align the housing 92 in the connector 22 along the connector axis 30. The sliding piston 40 and syringe 14 are inserted into the syringe end 22b and the vial 12 is mounted to the vial end 22a. The syringe 14 is urged from the initial position toward the vial end 22a until the syringe needle 94 pierces the tip cap 26 such that the tip of the syringe needle 94 is located in the receiving space 28 and the first end 92a of the housing 92 is in facing engagement with the second end 40b of the sliding piston 40. The syringe 14 is urged from this intermediate position toward the vial end 22a until the tips of the vial and vent needles 96, 98 penetrate the vial stopper 12a, creating a fluid passageway between the vial 12 and syringe 14 through the vial needle 96, the fluid cavity 92d and the vial needle 96. Fluid and/or medication may be drawn through the fluid passageway and the fluid filter 102, which generally filters contaminants or large objects as they flow through the cavity 92d. In addition, as a vacuum or a high pressure is formed in the vial, air is drawn into or expelled from the vial 12 through the vent needle 98 and this air passes through the air filter 100.

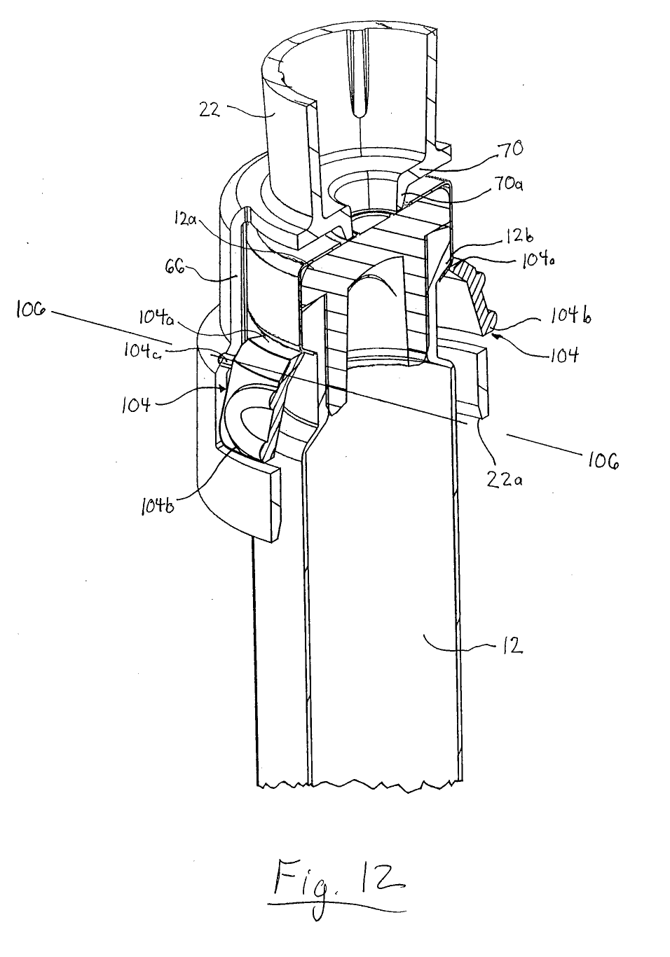

Referring to FIGS. 11 and 12, in the second preferred embodiment the vial mounting mechanism proximate the vial end 22a of the connector 22 includes a pair of spring feet 104 mounted in the lock windows 66. The spring feet 104 are preferably constructed of the same polymeric material as the connector 22 and are preferably pivotally mounted to the connector 22 on a pivot axis 106. Each of the spring feet 104 includes a grasping end 104a, a manipulating end 104b and a pair of pivot nubs 104c extending from sides of the feet 104 between the grasping and manipulating ends 104a, 104b. The spring feet 104 are biased toward a grasping position (FIGS. 11 and 12) where the grasping end 104a is proximate the connector axis 30 and may be pivoted to an extended position, where the grasping end 104a is radially spaced from the connector axis 30 such that the vial stopper 12a may be inserted into or removed from the vial end 22a. The spring feet 104 are not limited to the inclusion of the pivot nubs 104c and may be pivotally mounted to the connector 22 by a pivot shaft or a living hinge, as would be obvious to one having ordinary skill in the art. The pivot feet 104 of the second preferred embodiment is not limited to the above-described structure and may be comprised of nearly any structure that is able to releasably mount the vial 12 to the vial end 22a.

In operation, a user squeezes the manipulating ends 104b toward the connector axis 30 such that the spring feet 104 pivot about the pivot axis 106 and the vial 12 is inserted into the vial end 22a until the vial stopper 12a is in facing engagement with the vial ring 70a of the vial shoulder 70. The manipulating ends 104b are released and the bias force urges the spring feet 104 to pivot on the pivot axis 106. The spring feet 104 pivot until the grasping ends 104a engage the vial 12 beneath the peripheral rim 12b to secure the vial 12 to the connector 22 and align the vial 12 along the connector axis 30. The operation of the syringe safety device 10 then proceeds as was described above to mix and draw medication into the syringe 14. When the contents of the vial 12 are drawn into the syringe 14, the user again squeezes the manipulating ends 104b to release the vial 12 from the connector 22. Another vial 12 may then be inserted into and secured to the vial end 22a of the connector 22 for additional mixing and/or transferring of medication to and from the second vial 12 and the syringe 14. The spring feet 104 are particularly useful when a drug dose or mixture is contained in more than one vial 12 and both doses need to be injected into the patient through a single syringe 14.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A tip cap for a syringe that limits contact between the syringe and a needle tip wherein the needle tip penetrates the tip cap to one of dispense medication our of the syringe and draw medication into the syringe, the syringe including a luer cone extending from a barrel, the tip cap comprising:
a penetration wall; and
a generally cylindrical sleeve extending generally perpendicularly from the penetration wall adjacent a periphery of the penetration wall, the cylindrical sleeve including a radially inwardly extending locating shoulder on an inner surface located a predetermined distance from the penetration wall, the locating shoulder engaging an end of the luer cone in an operating position, the penetration wall, cylindrical sleeve and luer cone defining a receiving space within which the needle tip is positionable.

2. The tip cap of claim 1 wherein the tip cap is constructed of a self-healing, polymeric material.

3. The tip cap of claim 1 wherein the cylindrical sleeve includes a tip mouth, the tip mouth contacting a syringe luer lock which is mounted to the luer cone in the operating position, the contact between the tip mouth and the luer lock further positioning the tip cap relative to the end of the luer cone.

4. The tip cap of claim 1 wherein an inner surface of the cylindrical sleeve on an engaging portion of the sleeve tapers away from a connector axis.

5. The tip cap of claim 1 wherein the engaging portion is force fit onto the luer cone.
